(19) United States
(12) Patent Application Publication
(10) Pub. No.: US 2020/0170510 A1
(43) Pub. Date: Jun. 4, 2020

(54) DEVICE FOR SENSOR BASED MEDICATION DELIVERY BASED ON MOTOR, SENSORY, COGNITIVE AND PHYSIOLOGICAL FLUCTUATIONS

(71) Applicant: AppTek, Inc., McLean, VA (US)

(72) Inventors: Darius FERDOWS, Bethesda, MD (US); Yasar YAGHI, Arlington, VA (US); Mudar YAGHI, McLean, VA (US); Fernando PAGAN, McLean, VA (US); Charbel MOUSSA, Germantown, MD (US)

(73) Assignee: AppTek, Inc., McLean, VA (US)

(21) Appl. No.: 16/667,511

(22) Filed: Oct. 29, 2019

Related U.S. Application Data
(60) Provisional application No. 62/752,125, filed on Oct. 29, 2018.

(51) Int. Cl. A61B 5/00; G16H 20/13; G16H 40/67; A61B 5/16; A61B 5/11; A61M 5/172

(52) U.S. Cl. CPC A61B 5/0036; A61M 2205/04; G16H 40/67; A61B 5/0031; A61B 5/7475; A61B 5/163; A61B 5/0077; A61B 5/4023; A61B 5/162; A61B 5/112; A61M 5/1723; A61B 2562/0204; A61M 2205/505; A61M 2205/50; A61M 2205/52; A61M 2205/3523; A61M 2205/3303; A61M 2230/63; G16H 20/13

(57) ABSTRACT

A system and method dispense medication through a smart pill that holds at least one medication and a mobile device capable of communicating wirelessly with the smart pill. The mobile device includes a user interface for communicating with a patient and at least one application program that includes program instructions for measuring an aspect of a patient's physiology. A processor on the mobile device is configured to execute a program for monitoring and communicating with the smart pill, causing at least one of the applications to execute and test the patient, and on the basis of the outcome of the testing, issue a signal for timing and amount of medication release from the smart pill to the patient. The application programs may include a cognitive test, an eye test, a balance test, and a reaction test which may use the display, camera, speaker and microphone of the mobile device.

### FIELD OF THE INVENTION

The present invention relates generally to personalized drug delivery systems and methods and, more particularly, to automated, on demand, personalized drug delivery systems and methods that incorporate sensors, mobile tests and/or monitoring and are capable of producing time and dose specific drug delivery.

### BACKGROUND OF THE INVENTION

Systems for testing a patient's balance or gait and mental condition have been used in diagnosis of medical conditions. However, generally such techniques have been used to determine an overall course of treatment or dosages. Patients frequently find it difficult to maintain a course of treatment by taking medication on a schedule. Moreover, a particular schedule of dosages, even when followed, may not be optimal for a patient given day to day variations in a patient's physical activity or other variations in a patient's condition.

There is a need for medical systems and methods for delivering medication to patients that helps patients by delivering medication on time. There is a further need for a medication delivery system that is capable of delivering medication to patients in optimal doses in a personalized manner.

### SUMMARY OF THE INVENTION

According to an embodiment of the present invention, therapy includes a drug delivery vehicle in the form of a smart pill that may be ingested or implanted subcutaneously that communicates with a mobile device to control doses in time and/or amount. The pill may have sensors and a modem that enables communication with the mobile device. The mobile device may be used to control dosing by monitoring or testing the patient using sensors on the mobile device or the pill, cameras, and/or interactive mobile device sessions with the patient. The monitoring or tests may rely on sensors for kinetic detection of patient movement in three dimensional space to deliver medication in a time and dose specific manner.

According to one embodiment of the invention, a device and/or a portable sensor is used to detect motor, sensory, cognitive and physiological fluctuations and deliver medication in an automated, on demand personalized manner, in real time, to patients with a disease, such as a neurological, psychiatric and other brain diseases. The system will deliver medication via various methods, for example, a subcutaneous chip, smart pill or an implanted pump. The system may have the capability to control the delivery of more than one medication at various rates and in response to the patient's functional status.

According to another embodiment of the invention, a system or method will detect motor, sensory, cognitive, and physiological parameters that will allow for a personalized drug(s) delivery based on algorithms and/or artificial intelligence (“Al”) interpretations of the patient's physiological functions. Medications can be either standard of care or new experimental ones aimed at improving function and quality of life. In cases where disease can fluctuate from an “on” to “off States” or hyperkinetic to hypokinetic states, the system or method will respond to increase medication delivery or decrease medication delivery or release an entirely new medication other than the maintenance medication in response to these fluctuations. The system may capture data or information from both motor and non-motor symptoms and respond accordingly.

The system and method may include the following features. The technology for the medication delivery vehicle may be any delivery vehicle that can be safely deployed in the human body and controlled wirelessly or otherwise configured to respond to medical conditions and deliver medication in a personalized, time and dosage specific manner. For example, through subcutaneous or ingested medication smart pills may include a combination of one or more drugs that can be used to treat different symptoms. An example of a smart pill is one that would release levodopa alone or in combination with a dopamine agonist, or a monoamineoxidase inhibitor. These drugs would be stored in different physical layers of the pill and released concurrently or separately. Another example would be a pill that contains an acetylcholinesterase inhibitor as well as levodopa to concurrently or separately treat cognitive and motor symptoms. Smart pills could consist of polymer membranes, peristaltic pumps, and could be administered systemically, sublingually, or via surgical implantation. Additionally, a mobile device according to an embodiment of the invention could interface with a, subcutaneous, intraintestinal, or intra-thecal pump/device, as well as pacemaker - like devices (pulse generators) to deliver more individual therapies to treat motor and non - motor symptoms of patients.

A smart pill may be a Bluetooth enabled pill that releases one or more types of medication in response to electronic signals received by the Bluetooth system. The pill is able to communicate information regarding the release of medication, including timing of the release and the quantity of the release.

A system and method according to the present invention can communicate with the pill using Bluetooth. The system and method can also communicate with one or more sensors to help monitor and administer the release of medication through a series of tests. The system and method may be used on a mobile device, such as a smart phone or tablet computer. Through a mobile device which is connected to a pill wirelessly enabled with Bluetooth or another wireless protocol, a mobile device may be configured to trigger release of medication from the pill at personalized times and amounts based on prescriptions and cognitive tests, reaction tests, gait or balance tests, eye tracking, external factors, machine learning or any combination of the foregoing.

According to still another embodiment of the invention, a system for dispensing medication includes a smart pill that holds at least one medication for dispensing to a patient and a mobile device capable of communicating wirelessly with the smart pill. The mobile device includes at least one user interface system for communicating with a patient including a display, a microphone, a speaker and a camera and at least one sensor system such as an accelerometer for detecting movement. The device further includes a processor and a memory. The memory includes at least one application program that includes program instructions for measuring an aspect of a patient's physiology. The processor is configured to interact with the memory and at least one of the user interface systems to execute a program for monitoring and communicating with the smart pill, causing at least one of the applications to execute and test the patient, and on the basis of the outcome of the testing, issue a signal for timing and amount of medication to release from the smart pill to the patient.

The application programs may include a cognitive test, an eye test, a balance test, and a reaction test. Among other systems, the application programs may use the display, camera, speaker, microphone and accelerometer sensors of the mobile device. The signal to the smart pill may be a dosage schedule update or a command to the smart pill to release at least one medication in a respective dosage amount at a particular time. In addition, the outcome of the testing may be at least one score and the dosage amount and timing may be determined based on the at least one score resulting from the at least one test.

According to still another embodiment of the invention, a method for dispensing medication includes configuring a smart pill to hold at least one medication for dispensing and configuring a mobile device with at least one application program to communicate wirelessly with the smart pill. The method further includes monitoring and communicating with the smart pill using the at least one application and testing the user using at least one application program on the mobile device and then issuing a signal to the smart pill based on the outcome of the testing for timing and amount of medication release to the patient by the smart pill.

### BRIEF DESCRIPTION OF THE FIGURES

The above described features and advantages of the present invention will be more fully appreciated with reference to the appended drawing figures described below.

FIG. 1 depicts a functional block diagram of a smart pill in communication with a mobile device according to an embodiment of the invention.

FIG. 2 depicts a method of controlling dosing in a smart pill according to a cognitive test according to an embodiment of the invention.

FIG. 3 depicts a method of controlling dosing in a smart pill according to a reaction test according to an embodiment of the invention.

FIG. 4 depicts a method of controlling dosing in a smart pill according to a balance test according to an embodiment of the invention.

FIG. 5 depicts a method of controlling dosing in a smart pill according to an eye test according to an embodiment of the invention.

FIG. 6 depicts a system including a smart pill and a mobile device for controlling therapeutic dispensation of medication from the smart pill according to an embodiment of the invention.

### DETAILED DESCRIPTION

The present application claims priority to, an incorporates by reference herein the prior provisional patent application filed on Oct. 29, 2018 and entitled, “DEVICE FOR SENSOR BASED MEDICATION DELIVERY BASED ON MOTOR, SENSORY, COGNITIVE AND PHYSIOLOGICAL FLUCTUATIONS” and having U.S. Provisional patent application No. 62/752,125.

According to an embodiment of the present invention, therapy includes a drug delivery vehicle in the form of a smart pill that may be ingested or implanted subcutaneously that communicates with a mobile device to control doses in time and/or amount. The pill may have sensors and a modem that enables communication with the mobile device. The mobile device may be used to control dosing by monitoring or testing the patient using on sensors on the mobile device or the pill, cameras, and/or interactive mobile device sessions with the patient. The monitoring or tests may rely on sensors for kinetic detection of patient movement in three dimensional space to deliver medication in a time and dose specific manner.

According to one embodiment of the invention, a device and/or a portable sensor is used to detect motor, sensory, cognitive and physiological fluctuations and deliver medication in an automated on demand personalized manner, in real time, to patients with a disease, such as a neurological, psychiatric and other brain diseases. The system will deliver medication via various methods, for example, a subcutaneous chip, smart pill or an implanted pump. The system may have the capability to control the delivery of more than one medication at various rates and in response to the patient's functional status.

The system may release medication via various methods, e.g. a subcutaneous chip, smart pill or an implanted pump as described below. For example, implantable devices include but are not limited to those that may deliver medication intravenously, intramuscularly, intraperitoneally, intraventricularly, intrainstinally, intrathecally and might administer drug, antibody therapy, or gene delivery (including but not limited to anti sense nucleaotide, si-RNA, Sh-RNA, micro-RNA, DNA, delivery). The system may have the capability to control the delivery of more than one medication at various rates and in response to the patient's functional status. The system is capable of detecting motor, sensory, cognitive, and physiological parameters that will allow for a personalized drug (s) delivery based on algorithms and Al interpretations of the patient's physiological functions. Medications can be either standard of care or new experimental ones aimed at improving function and quality of life. In cases where disease can fluctuate from an “on” to “off States” or hyperkinetic to hypokinetic states, the system will respond to increase medication delivery or decrease medication delivery or release an entirely new medication other than the maintenance medication. The system will capture data or information from both motor and non-motor symptoms and respond accordingly

Tests and monitoring implemented on the mobile device that may be used to control the pill and the release of medication from the pill are described briefly below.

### 1. Cognitive Tests

Using speech recognition, memory tests of a patient may be conducted through a mobile application on the patient's mobile (or other) device. Speech patterns, tone, volume and other measurements may be made in addition to administering the memory test using a touch screen display, speaker, buttons, microphone and/or other input/out devices on the mobile device. Measurements and a score determined during the test may be used to determine when and whether to release medication within the smart pill that has been ingested or implanted underneath the skin (subcutaneous) or in what amount.

### 2. Reaction Tests

In a mobile application, users may be taken through a tapping exercise to measure reaction time. The exercise may be presented to the user through a display or speakers of the mobile device and the input from the user may be collected through pressing buttons or through a touch screen interface on the mobile device. Measuring response time of the user may be used to determine a variety of neurological issues.

### 3. Gait/Balance Tests or Monitoring

A general balance test and gait test may be performed on a mobile device. The accelerometer and gyroscope measurements may be used by an application running on the mobile device to determine balance and gait which will in turn determine successful or unsuccessful tests. Motor skills may be tested here through pose estimation technologies performed through a mobile device. The results of the gait or balance test may be used to determine a score or otherwise to determine the timing or amount of dosage if any required based on the test.

### 4. Eye Tracking

Measuring eye movements, pupil size, and pupil dilation can help determine a variety of neurological issues which may be occurring. Using deep learning data, certain assumptions can be made from the biology of the eye. Using an eye tracking application on a mobile device, such measurements may be made by the camera on the mobile device an used to determine a neurological condition and in turn a score or other test result may be used to control the timing or amount of dosing of medications contained in a smart pill.

### 5. External Factors (Physiological and Demographics)

Nutrition, gender, weight, family history, other medications taken, and other factors may be accounted for to better determine the release of medication dosage. This information may be stored in the mobile device and pushed to medical providers through a portal system. This information is also useful in determining how the body reacts to different medication.

### 6. Specialty Testing or Monitoring

The smart pill and methods described herein are especially useful to address neurological issues. However, the methods may be applied virtual all areas involving correct medical dosages such as ADHD, mental disorders, learning disabilities, and many other areas. These would also include adult and pediatric neurological diseases. Physiological changes resulting from the medication may also be measured. Drugs may be FDA approved for immediate use of this technology or may be still experimental for future patient use.

### 7. Machine Learning

As more data is collected one or more patients, the system and method may develop a database of medication release and its physiological effects on one or more patients. Having additional data is useful, allowing the system and method to become smarter over time and administer time release dosages in a more personalized and efficient manner. This technology may combine sensors and delivery of medication in real-time drug administration.

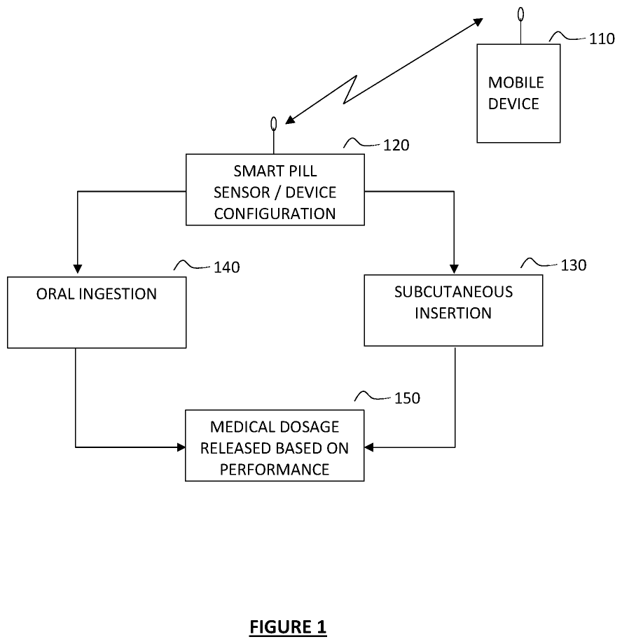

The operation of the system and methods in various embodiments are described in additional detail with reference to the figures. FIG. 1 depicts a functional block diagram of a system 100, according to an embodiment of the invention, for delivering doses of medication to a patient based on testing or monitoring. Referring to FIG. 1, a mobile device 110 is in communication with a smart pill sensor or device 120. The mobile device may be a typical smart phone, tablet, personal data assistant, laptop or any other device capable of wireless communication. The smart pill also includes the ability to wirelessly communicate with the mobile device and further includes the ability to be configured to accept control signals from the mobile device and release medication at particular times and in particular amounts. As described later in the application, the pill may include more than one medication reservoir, one or more pumps and electronics to activate the pumps to release one or more medications to the body at particular times and in particular doses. The smart pill 120 may be placed in the body through oral ingestion 140 or through subcutaneous insertion 130 by a medical service provider. During use, the smart pill and mobile device communicate to ensure that medical dosages are released in amounts and at times based on the patient's performance on tests and/or monitoring by the mobile device. This ensures both patient compliance with a course of treatment, but also ensures that the medication is taken at optimum times for the patient based on the patient's actual need for the medication.

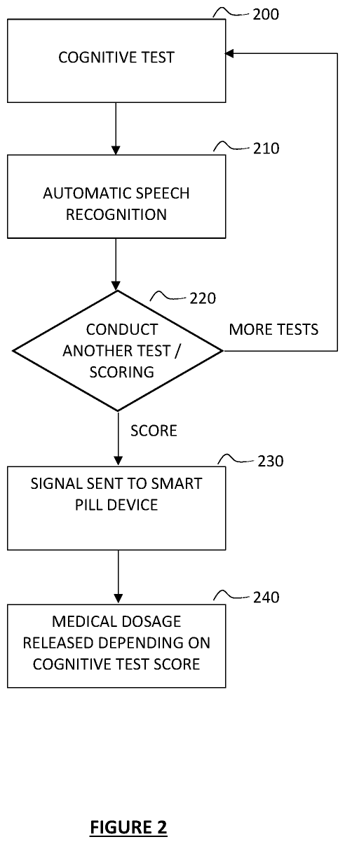

FIG. 2 depicts a flow diagram for controlling an exemplary cognitive test according to an embodiment of the invention. Referring to FIG. 2, in step 200, a cognitive test may be conducted on a mobile device. The cognitive test may be conducted using the user interfaces of the mobile device to communicate with the user or patient. For example, in step 210 speech recognition functionality within the mobile device may be used to conduct memory tests through a mobile application running on the mobile device. The mobile device interacts with the patient that has ingested or otherwise is using a subcutaneously inserted smart pill. In step 220, the application may determine the results of a test, conduct another test or determine scoring for one or more completed tests. Based on the score(s) determined in step 220, in step 230 the timing and amount of the release of medication in the smart pill may be determined and a signal transmitted to the smart pill. The frequency of the tests may be determined in advance by a supervising physician and thereafter conducted in an automated manner by the mobile device to modify the timing and dosage of medication to be delivered within predetermined parameters set by the physician. In addition, the Speech patterns, tone, volume and other measurements can help determine the release of medication that has been ingested or implanted underneath the skin (subcutaneous). For example, the detection of slurred speech or differences in tonality may be scored in order to and used as factors in determining timing and amount of medication dosages to be released by the smart pill. In addition, an overall score of the cognitive test may be used to determine whether and how much medication should be released. In step 240, the smart pill may receive the signal from the mobile device. The signal may include timing and dosage release information. Thereafter the smart pill may release medication to the patient's body at a particular dosage level and at a particular time based on the signal received from the mobile device. The smart pill may be programmed to release medication according to a predetermined schedule that is modified by the signal from the mobile phone. Or the smart pill may be programmed to not release any medication until the signal is received from the mobile device. The mobile device may also send updates in dosage schedule to the smart pill based on the results of the cognitive or other tests or monitoring of the patient described herein.

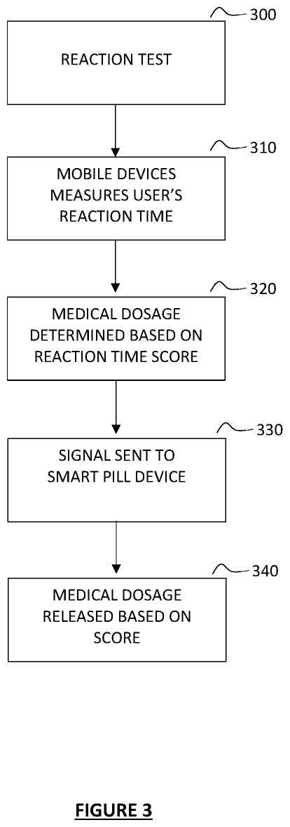

FIG. 3 depicts a method of controlling dosage from a smart pill according to a reaction test according to an embodiment of the present invention. Referring to FIG. 3, in step 300, a reaction test may be conducted on a mobile device. The reaction test may be conducted using the user interfaces of the mobile device to communicate with the user or patient. For example, in step 310 the user's reaction time may be determined by measuring the amount of time it takes for the user of a mobile device to respond to prompts from the mobile device. Users of a mobile application, for example, may be taken through tapping exercises to measure reaction time. Measuring response time can be used to determine a variety of neurological issues. By, for example, creating a stimulus to which the patient and user of a mobile device needs to respond by tapping a touch screen of the mobile device, a test may be administered to the patient. The response time may be determined and then compared to averages among a patient population or averages for the specific patient being tested. A score or other test result information may be determined in step 310 based on the user's responses. Based on the score(s) determined in step 310, in step 320 the timing and dosage amount for release of one or more medications from a smart pill that the user has ingested or that is implanted may be determined. In step 330 a signal transmitted to the smart pill that may include timing and dosage information for each medication to be released to the patient. The frequency of the tests may be determined in advance by a supervising physician and thereafter conducted in an automated manner by the mobile device to modify the timing and dosage of medication to be delivered within predetermined parameters set by the physician. Thereafter in step 340 the smart pill may release medication to the patient's body at a particular dosage level and at a particular time based on the signal received from the mobile device. The smart pill may be programmed to release medication according to a predetermined schedule that is modified by the signal from the mobile phone based on the patient's reaction time. Or the smart pill may be programmed to not release any medication until the signal is received from the mobile device. The mobile device may also send updates in dosage schedule to the smart pill based on the results of the reaction test or other tests or monitoring of the patient described herein.

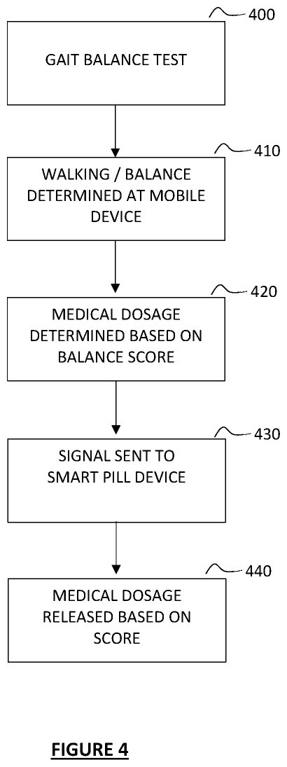

FIG. 4 depicts a method of controlling dosage from a smart pill according to a gait or balance test according to an embodiment of the present invention. Referring to FIG. 4, in step 400, a gait or balance test may be conducted on a mobile device. The gait or balance test may be conducted using sensors of the mobile device and/or the user interfaces of the mobile device when communication with the user or patient is desired. In step 410, the gait or balance tests may be performed continuously by the mobile device to monitor the patient's balance or gait over time to detect degradation and therefore determine optimal timing and amounts of medication. Alternatively, the balance or gait testing may be performed at intervals or through interaction between the mobile device and the patient according to a predetermined schedule, at an adaptable schedule or at the prompting of the patient. In general, balance and/or gait tests may be performed using an accelerometer or gyroscope measurements using sensors found on typical mobile devices, such as smartphones or tablets. The mobile device, based on well-known analyses of sensor data may determine successful or unsuccessful tests and score them with for example a pass fail score or a score that is based on a degree of imbalance or gait. Motor skills may be tested here through pose estimation technologies performed through a mobile device. In general, the sensors within a mobile device may be used when the patient walks and holds the device in order to make motion measurements for the patient. In step 420, Information about the movement, including gait, may then form the basis, in whole or in part, and potentially combined with other information to determine whether or not medication should be dispensed to a patient using the smart pill, and in what amount and for what duration. In step 430 a signal transmitted to the smart pill that may include timing and dosage information for each medication to be released to the patient determined by testing the user's balance or gait. The frequency of the tests may be determined in advance by a supervising physician and thereafter conducted in an automated manner by the mobile device to modify the timing and dosage of medication to be delivered within predetermined parameters set by the physician. Thereafter in step 440 the smart pill may release medication to the patient's body at a particular dosage level and at a particular time based on the signal received from the mobile device. The smart pill may be programmed to release medication according to a predetermined schedule that is modified by the signal from the mobile phone based on the patient's balance or gait test results. Or the smart pill may be programmed to not release any medication until the signal is received from the mobile device. The mobile device may also send updates in dosage schedule to the smart pill based on the results of the balance or gait test or other tests or monitoring of the patient described herein.

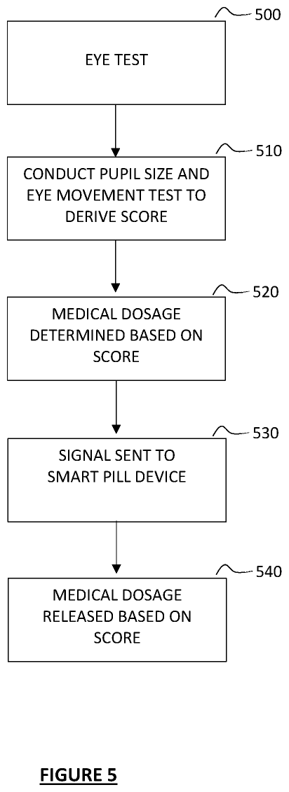

FIG. 5 depicts a method of controlling dosage from a smart pill according to an eye test according to an embodiment of the present invention. Referring to FIG. 5, in step 500, an eye test may be conducted on a mobile device using a camera on the mobile device. Measuring eye movements, pupil size, and pupil dilation can help determine a variety of neurological issues which may be occurring in a patient or user. Using a mobile device to photograph and measure pupil size, dilation and movement can create data used to control the dispensation of medicine. Depending on the data from the mobile device, and whether certain thresholds are met or exceeded, a signal can be sent to the pill to release medication, release it at a particular time or in a particular amount, or otherwise at a particular rate and time. The eye test may be conducted using a camera on the mobile device and/or the user interfaces of the mobile device when communication with the user or patient is desired. The eye tests may be performed continuously by the mobile device to monitor the patient's pupils in size and dilation and eye tracking. Alternatively, the eye testing may be performed at intervals or through interaction between the mobile device and the patient according to a predetermined schedule, at an adaptable schedule or at the prompting of the patient. In general, eye tests may be performed using the phone's camera and interactive display to facilitate capturing the user's eyes and pupils and to prompt the patient to move the patient's eyes or otherwise face the camera to facilitate testing. The mobile device, based on analyses of camera data may determine successful or unsuccessful tests and score them with, for example, a pass fail score or a score that is based on a degree of delay or other irregularity in eye tracking, or degree of pupil size or dilation as compared to norms for the user or patient or compared with averages for a patient population. Pass or fail may relate to when pupil size or dilation exceeds or is below predetermined thresholds or when eye tracking parameters exceed or are below predetermined thresholds. In general, the camera within a mobile device may be used when the patient holds the device pointed at the user's face in order to make pupil or eye motion measurements for the patient. In step 520, Information about the eye tests may then form the basis, in whole or in part, and potentially combined with other information to determine whether or not medication should be dispensed to a patient using the smart pill, and in what amount and for what duration. In step 530 a signal transmitted to the smart pill that may include timing and dosage information for each medication to be released to the patient determined by eye testing. The frequency of the tests may be determined in advance by a supervising physician and thereafter conducted in an automated manner by the mobile device to modify the timing and dosage of medication to be delivered within predetermined parameters set by the physician. Thereafter in step 540 the smart pill may release medication to the patient's body at a particular dosage level and at a particular time based on the signal received from the mobile device. The smart pill may be programmed to release medication according to a predetermined schedule that is modified by the signal from the mobile device based on the patient's eye test results. Or the smart pill may be programmed to not release any medication until the signal is received from the mobile device. The mobile device may also send updates in dosage schedule to the smart pill based on the results of the balance or gait test or other tests or monitoring of the patient described herein.

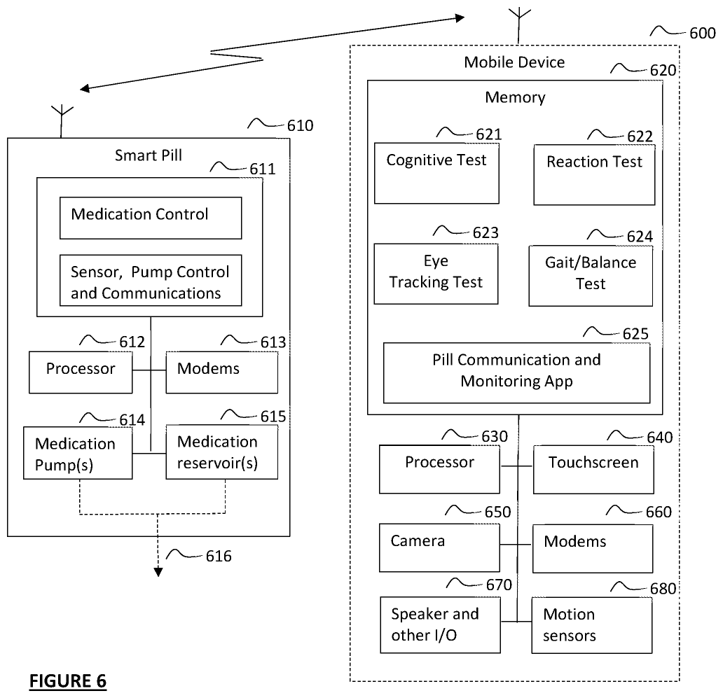

A system for controlling a smart pill and monitoring a patient according to an embodiment of the invention is shown in FIG. 6. The mobile device may be a smart card, tablet computer or any other kind of computer capable of communicating with the smart pill, for example wirelessly via modem(s) 660 using the Bluetooth protocol, wi-fi, near field communications (NFC). Any other wireless or other technology may be used for such communications.

The mobile device 600 includes a processor 630 coupled to a memory 620, a camera 650, a touchscreen or other display 640 and input or input/output devices including a speaker and microphone 670, modem(s) 660 and sensors 680 such as accelerometers and other sensors including but not limited to those found in smart phones and tablets for determining position and movement. The memory 620 stores data and program instructions corresponding to the methods described herein, which may include the applications for performing a cognitive test 621, a reaction test 622, an eye tracking test 623 and/or a gait and balance test 624 on the mobile device or smart phone. The processor accesses the memory and executes the program instructions corresponding to the methods and uses the camera, sensors and other devices as necessary to perform the tests. The memory may also include a pill communication and monitoring application 625 that communicates signals to the smart pill including schedules such as timing of dosage, amount of dosage for each medication to be released. The application 625 may also facilitate communicating schedule updates to the smart pill, receiving data from the smart pill on status of the smart pill and administration of the dosages. The application 625 may also give an instruction to initiate a dose at a particular time in a particular amount or to immediately initiate a dose of a particular amount for one or more medications. In addition, in some embodiments, the pill communication and monitoring application may also receive data from sensors on the pill that may be used in determining dosage timing and amounts.

When a patient ingests or otherwise is equipped with a smart pill and uses a mobile device, the mobile device tracks the administration of the medication or medications by the smart pill in the app 625 and is able to note changes in balance or gait, speech patterns, reactions and/or eye tracking or dilation through the apps 621, 622, 623, 624 or other similar applications designed to test a physiological aspects of the patient and make dose timing and size determinations for medications to be released to the patient. The mobile device 600 may signal to the patient to take a test to determine whether additional medication is needed or the patient may choose to activate an application to take a test to determine if more medication needs to be dispensed. Alternatively, the testing may be done continuously or in an automated manner or may be triggered by physiological events or sensed in the patient by one or more sensors described herein.

If more medication needs to be dispensed, the pill communication and monitoring app 625, sends a signal to the smart pill to dispense the medication. The communication and monitoring app may be equipped to determine proper dosing based on factors, including age, height, weight, male/female, type of disease, symptoms, etc. and particular parameters from a treating physician including dosage parameters per day or per week or symptom driven dosages associated with testing conditions (cognitive, reflex, eye, balance or other conditions) falling outside of predetermined parameters. It may be equipped as well to determine a dose based on particular feedback from the apps on the mobile device.

The smart pill 610 may illustratively include a memory 611 coupled to a processor 612, that is in turn coupled with modem(s) 613, medication pump(s) 614 and medication reservoirs 615. The memory may include program instructions that when executed by the processor cause the smart pill to communicate with the mobile device via the modem, control the pump(s) and control the reservoirs to ensure that the pill delivers correct doses of one or more medications to the patient at the correct time and in the correct amounts. For example, a medication control application and a sensor and pump control application may include the above described functionality for controlling the smart pill and the dispensation of treatment. In addition, the smart pill may include pathways between the pump(s) 614 and the reservoirs 615 that are in fluid communication with the patient's body that enable the medicine(s) in the reservoir to be dispensed to the patient.

The smart pill may be initially configured to communicate in a secure manner with the mobile device through Bluetooth pairing or through another wireless protocol that uses passwords or other forms of authentication. The smart pill may further be configured with a default schedule for releasing the medication that may be updated by the mobile device signaling. The mobile device 600 may send to the smart pill schedule updates, new default schedules or one off dosing instructions. The schedules may include timing and dosage amounts to be administered by the smart pill for each medication capable of dispensation by the smart pill. The memories described herein may include non-volatile and volatile memory and the memory may store a record of the history of all doses administered by the smart pill.

While particular features have been illustrated and described herein, it will be understood by those having ordinary skill in the art that changes may be made to those embodiments without departing from the spirit and scope of the invention.

What is claimed is:

1. A system for dispensing medication, comprising:
a smart pill that holds at least one medication for dispensing;
a mobile device capable of communicating wirelessly with the smart pill, and including at least one user interface system for communicating with a patient, the mobile device including a processor and a memory and at least one sensor system;
the memory including at least one an application program that includes program instructions for measuring an aspect of a patient's physiology; and
the processor configured to interact with the memory and at least one of the user interface systems and executing a program for monitoring and communicating with the smart pill, the mobile device causing at least one of the applications to execute and test the patient, and on the basis of the outcome of the testing, issue a signal for timing and amount of medication release to the patient via the smart pill.

2. The system according to claim 1, wherein the application program includes a cognitive test and the systems include a display and a microphone of the mobile device.

3. The system according to claim 1, wherein the application program includes an eye tracking test and the systems include a display and a camera of the mobile device.

4. The system according to claim 1, wherein the application program includes a balance test and the systems include a sensor on the mobile device.

5. The system according to claim 1, wherein the application program includes a reaction test and the systems include a display.

6. The system according to 1, wherein the application program includes at least two of a cognitive test, an eye tracking test, a gait/balance test, and a reaction test.

7. The system according to claim 6, wherein the signal is a dosage schedule update.

8. The system according to claim 7, wherein the signal is a command to the smart pill to release at least one medication in a dosage amount at a particular time.

9. The system according to claim 8, wherein the outcome of the testing is at least one score and the dosage amount is determined based on the at least one score resulting from the at least one test.

10. A method for dispensing medication, comprising:
configuring a smart pill to hold at least one medication for dispensing;
configuring a mobile device with at least one application program to communicate wirelessly with the smart pill;
monitoring and communicating with the smart pill using the at least one application,
testing the user using at least one application program on the mobile device to test the patient,
issuing a signal to the smart pill based on the outcome of the testing for timing and amount of medication release to the patient by the smart pill.

11. The method according to claim 10, wherein the at least one application program includes a cognitive test that uses a display and a microphone of the mobile device.

12. The method according to claim 10, wherein the at least one application program includes an eye tracking test that uses a display and a camera of the mobile device.

13. The method according to claim 10, wherein the at least one application program includes a balance test that uses a sensor on the mobile device.

14. The method according to claim 10, wherein the at least one application program includes a reaction test and uses the display of the mobile device.

15. The method according to 10, wherein the at least one application program includes at least two of a cognitive test, an eye tracking test, a gait/balance test, and a reaction test.

16. The method according to claim 15, wherein the signal is a dosage schedule update.

17. The method according to claim 16, wherein the signal is a command to the smart pill to release the at least one medication in a respective dosage amount at a particular time.

18. The method according to claim 17, wherein the outcome of the testing is at least one score and the dosage amount is determined based on the at least one score resulting from the at least one test.
